(19) United States
(12) Plant Patent Application Publication
(10) Pub. No.: US 2018/0235127 P1
(43) Pub. Date: Aug. 16, 2018

(54) CLEMATIS PLANT NAMED 'EVIPO079'

(50) ;

(71) Applicants: Mogens Nyegaard Olesen, Fredensborg (DK); Raymond J. Evison, St. Sampsons (GG)

(72) Inventors: Mogens Nyegaard Olesen, Fredensborg (DK); Raymond J. Evison, St. Sampsons (GG)

(21) Appl. No.: 15/330,280

(22) Filed: Sep. 6, 2016

(51) Int. Cl. A01H 6/72

(52) U.S. Cl. PLT/228

(57) ABSTRACT

A new Clematis plant with a compact growth habit, profuse, red flowers, and continuous summer flowering. The variety successfully propagates from softwood cuttings and is suitable for cultivation in commercial nursery culture. This new and distinct variety has shown to be uniform and stable in the resulting generations from asexual propagation from vegetative cuttings.

Botanical classification:

Genus: Clematis.

Species: viticella.

Variety denomination: ‘Evipo079’.

### SUMMARY OF THE CLAIMED PLANT

The present invention constitutes a new and distinct variety of Clematis plant which originated from a controlled crossing between the female seed parent, an un-named seedling, and the male pollen parent, an un-named seedling. Both parent varieties are non-patented.

The two parents were crossed during the summer of 2005 and the resulting seeds were planted the following winter in a controlled environment in Guernsey, Channel Islands, United Kingdom. The new variety named ‘Evipo079’ originated as a single seedling from the stated cross.

The new clematis plant may be distinguished from its female seed parent and male pollen parent by the following characteristics. The female seed parent has red purple tepals while the new variety has red tepals. The male seed parent has violet tepals while the new variety has red tepals.

The objective of the hybridization of this clematis plant was to create a new and distinct variety for nursery culture with unique qualities such as:

1. Uniform and abundant red flowers;

2. Vigorous and compact growth, making the variety suitable for container culture; and

3. Improved disease resistance.

This combination of qualities was lacking in clematis plants that were in commercial cultivation and the qualities have been substantially achieved in the new variety.

‘Evipo079’ was selected by Mogens N. Olesen and Raymond J. Evison in their clematis development program in the Channel Islands, United Kingdom in 2006. Asexual reproduction of ‘Evipo079’ by means of vegetative cuttings and traditional layering was first performed by Mogens N. Olesen and Raymond J. Evison in the nursery during the summer of 2006. This initial and subsequent asexual propagations have demonstrated that the characteristics of ‘Evipo079’ are true to type and are transmitted from one generation to the next.

### BRIEF DESCRIPTION OF THE DRAWING

The accompanying color illustrations show as true as is reasonably possible to obtain in color photographs of this type the typical characteristics of the buds, flowers, leaves, and stems, of ‘Evipo079’.

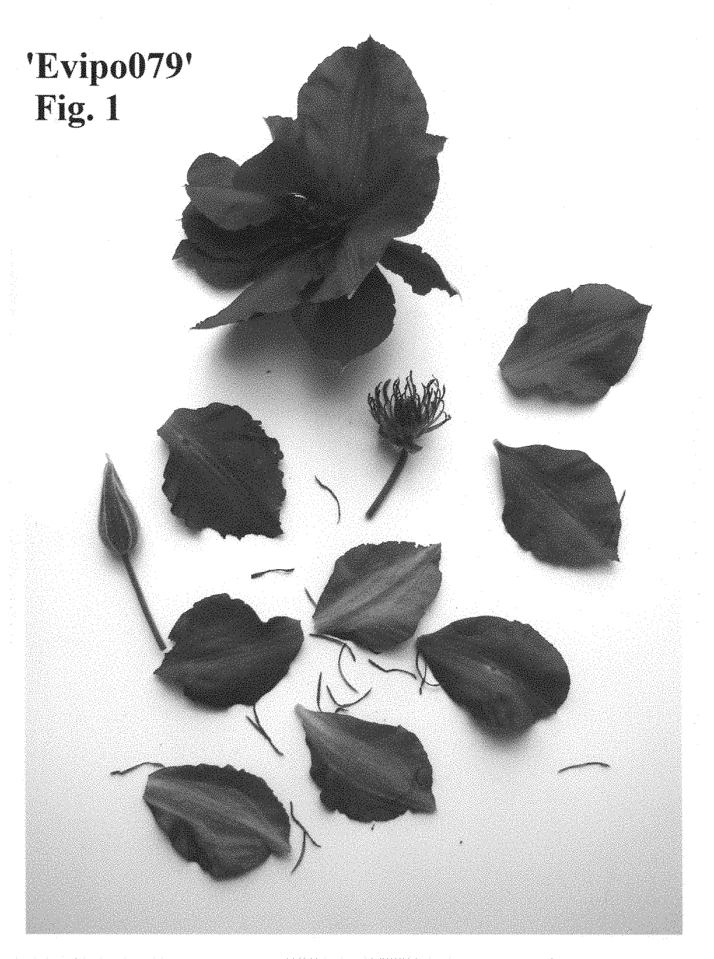

Specifically illustrated in FIG. 1 of the drawings is an open flower, sepals detached, reproductive flower parts, and a flower bud.

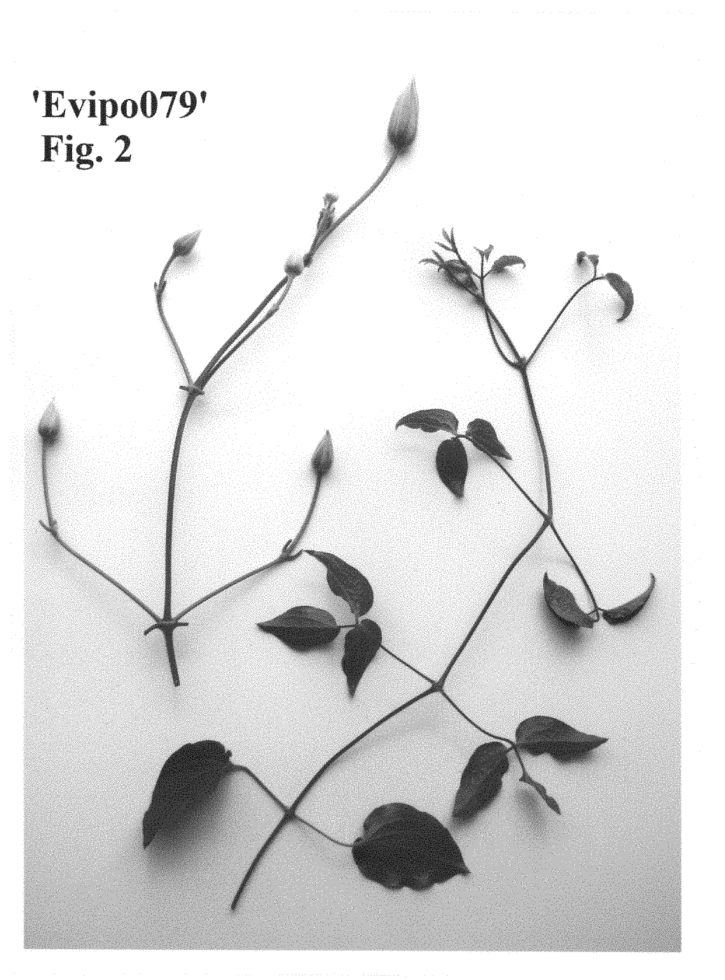

Specifically illustrated in FIG. 2 are stems showing leaves and attachment of flower buds. Plants shown are 2 years of age.

### DETAILED DESCRIPTION OF THE VARIETY

The following is a detailed description of ‘Evipo079’, as observed in its growth throughout the flowering period in Marion County Oreg. Observed plants were cultivated for a period of 24 months in 2 liter containers. Certain phenotypical characteristics of the variety may vary under different environmental, cultural, agronomic, seasonal, and climatic conditions. Color references are made using The Royal Horticultural Society (London, England) Colour Chart, 2001, except where common terms of color are used.

For a comparison, several physical characteristics of the clematis variety ‘Evipo016’ described and illustrated in U.S. Plant Pat. No. 21,583 are compared to ‘Evipo079’ in Chart 1.

CHART 1

| | ‘Evipo079’ | ‘Evipo016’ |
|---|---|---|
| Flower diameter | 115 mm | 130-185 min |
| Tepal upper | Red-Purple | Red Group 53A |
| surface upon | Group 59A | |
| opening | | |
| Tepal count | 8 | 6 |

Flower and flower bud:
   Blooming habit.—Recurrent. The natural flowering period is generally from May to September.
   Flower bud.—Size: Normally 33 mm in length. Bud diameter is 13 mm. Bud form: Elliptic with broad base. Bud color: At ¼ opening Greyed-Purple Group N186C. Texture: Highly pubescent.
   Pedicel.—Surface Texture: Slightly pubescent. Length: On average 65 mm in length with 30 mm diameter. Color: Yellow-Green Group 144A. Strength: Moderately strong.
   Receptacle.—Surface Texture: Lightly pubescent. Shape: Broad funnel. Size: 2 mm (h)×2 mm (w). Color: Yellow-Green Group 144A.
   Flower arrangement.—Location on vine: New and old growth. Borne: Singly at the terminal and axillary buds along the branch.
   Flower bloom.—Size: On average, flowers are 115 mm in diameter and 20 mm in depth. Profile: Open flowers are flat. Fragrance: None. Lasting Quality: Flowers normally remain up to 10 days on the plant.
   Tepals.—Tepal Color: Upon opening, the upper surface is Red-Purple Group 59A. The lower surface is Greyed-Purple Group 187C with intonations of Greyed-Purple Group 186A. Quantity: Normally 8 tepals. Size: 60 mm in length by 40 mm wide. Shape: Individual tepal shape is ovate. The tepal apex is acuminate. The tepal base is acute. Apex Recurvature: None. Tepal Cross section: Flat. Margins: Medium undulations of margin observed. Persistence: Tepals drop off cleanly.
   Reproductive organs.—Arrangement: Open. Pollen: None observed. Anthers: Size: 8 mm in length. Color: Purple Group N77A. Quantity: On average, 20. Filaments: Color: Purple Group 77B. Length: 10 mm. Pistils: Quantity: On average, 15. Stigmas: Inferior in location relative to the length of the filaments and the height of the anthers. Color: Greyed-Purple Group 186B. Styles: Color: Greyed-White Group 157B. Length: 9 mm.
Plant:
   Plant form.—Climbing.
   Plant growth.—Moderately vigorous.
   Size.—Seasons growth attains 100 cm in height. Average spread is 50 cm.
   Stems.—Color: Juvenile stems are Yellow-Green Group 144A. Mature stems are Yellow-Green Group 144A and Greyed-Purple Group 187A. Internodes: On average, 85 mm between nodes. Length: Normally 45 cm from the base of the plant to the flowering portion of the stem. Diameter: Normally 2 to 3 mm. Texture: Mature stems are generally smooth and lightly ribbed.
   Plant foliage.—Leaf characteristics: Deciduous. Arrangement: Simple and trifoliate. Leaf Size: Simple leaves are 50 mm (l)×45 mm (w). Compound trifoliate leaves are 80 mm (l)×70 mm (w). Terminal leaflets on trifoliate leaves are 40 mm (l)×19 mm (w). Abundance: On average 1 leaf per 10 cm of stem. Leaf Color: Juvenile upper. Juvenile lower. Mature upper Green Group 138A. Mature lower Green Group 139C. Stipules: Absent. Petioles: Size: Normally 40 to 45 mm in length by mm diameter. Texture: Smooth. Color: Yellow-Green Group 144A and Greyed-Red Group 181A. Petioloule: Size: Normally 10 mm in length by 2 mm diameter. Texture: Smooth. Color: Yellow-Green Group 144A and Greyed-Red Group 181A. Leaflet Shape: Generally ovate. The base is oblique. The apex is acute. Margin: Entire. Surface: The upper side is smooth. The lower side is smooth. Thickness: Average. Glossiness: Moderately glossy.
   Disease resistance.—Subject to any disease that normally attacks the species. However the variety is more tolerant to clematis wilt, Ascochyta clematidina, than some clematis known to the inventors.
   Cold hardiness.—The variety is tolerant to USDA Hardiness Zone 6.
   Heat tolerance.—The variety has been found to be suitable for climate conditions found in the American Horticulture Society heat zone 7.

We claim:

1. A new and distinct variety of clematis plant named ‘Evipo079’, substantially as described and illustrated , due to its abundant red flowers with good keepability, attractive long lasting foliage and compact growth, year round flowering under glasshouse conditions, suitability for production from softwood cuttings in pots, durable flowers and foliage which make the variety suitable for distribution in the floral industry.
